(19) United States
(12) Patent Application Publication
(10) Pub. No.: US 2006/0142304 A1
(43) Pub. Date: Jun. 29, 2006

(54) Method for treating or preventing pruritic and neurogenic skin disorders

(76) Inventors: Michael Southall, Lawrenceville, NJ (US); Frank Liebel, Bridgewater, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/315,773

(22) Filed: Dec. 22, 2005

Related U.S. Application Data
(60) Provisional application No. 60/639,382, filed on Dec. 27, 2004.

(51) Int. Cl. A61K 31/496; A61K 31/4178

(52) U.S. Cl. 514/254.07; 514/397

(57) ABSTRACT

This invention relates to methods of treatments, compositions, and kits for providing rapid relief to the user for pruritic and neurogenic skin disorders. The invention also provides methods of treatments, compositions, and kits for preventing such conditions.

### BACKGROUND OF THE INVENTION

The sensation of itch is one of the most common skin problems experienced by humans and animals. Itch can be defined as a sensation that provokes the desire to scratch the site from which the sensation originates. All skin contains sensory nerves, which can transmit itch or other similar sensory impulses in response to chemical irritation, environmental exposure or disease processes. No matter what the ultimate cause of itch, the sensation experienced is the same and provokes the desire to scratch.

Many people suffer from different skin conditions that result in itching, pain and general discomfort. Some of these conditions are caused by many of the skin or topical products now commercially available.

Chemical irritation is quite common. For example, many ingredients used in topical products are known irritants or are potentially irritating. Additionally, many topical products include active ingredients, including chemicals that may also be classified as drugs, may also produce irritation when applied to the skin or mucous membranes. This irritation may result in itching.

Environmental influences may also affect adversely the skin's barrier function. Extremes of humidity, for example, can greatly increase irritation from topically-applied products. A very common condition due to low humidity is termed “winter itch” in which the very low humidity characteristics of many cold climates (particularly when accompanied by indoor heating) or long exposure to refrigerated air from air conditioners in the summer produces itchy skin—especially in older people—which can exacerbate the irritating effects of topical products. Additionally, cleansing products including soaps and detergents, personal hygiene products such as shaving creams and other products which remove some of the skin's protective lipids and/or secretions may produce irritation. Personal hygiene activities such as shaving, waxing, skin peeling and exfoliating may also affect the skin's barrier function.

Some irritation and itching may be caused by skin diseases or conditions. Such conditions include eczema, psoriasis, acne, rosacea, contact irritant dermatitis, atopic dermatitis, allergic dermatitis, sunlight-induced dermatoses and dry skin. Other skin conditions that may result in itching include post-infection scarring, actions such as surgery (scarring including episiotomies), burns, hemorrhoids, insect and animal bites, stings, and skin conditions associated with endocrine and metabolic disorders such as. hyperthyroidism, hypothyroidism, diabetes, cholestasis, and uremia.

Fungal infections, including yeast infections, are infections, which are extremely uncomfortable and hard to successfully treat at times. Symptoms include itching, burning and if the infection is vaginal, an unpleasant odor and discharge may occur. One such infection is a vaginal yeast infection caused by Candida albicans. One preferred treatment for this infection is the topical use of sertaconazole as described in U.S. Pat. No. 5,135,943. Other treatments include azoles such as imidazoles and more specifically, miconazole nitrate, clotrimazole, econazole, albaconazole, ravuconazole, saperconazole, terconazole, ketoconazole, butaconazole, tioconazole, fluconazole, secnidazole, metronidazole, vericonazole, fenticonazole, sertaconazole, posaconazole, bifonazole, oxiconazole, sulconazole, elubiol, vorconazole, isoconazole, flutrimazole and their pharmaceutically acceptable salts and the like.

While many others have tried to address the causes of itching and to relieve the sensation/desire to itch, there has now been discovered a new way to treat itching using sertaconazole, a drug typically used for treating fungal, namely yeast infections.

### SUMMARY OF THE INVENTION

A method of treating a pruritis skin condition comprising an affected skin area that itches, said method comprising the steps of applying to said affected skin area a composition comprising sertaconazole and wherein said affected skin area is not infected. For the sake of clarity, the term “sertaconazole” as used herein refers to sertaconazole, its esters and its salts.

A method of treating an inflamed skin condition comprising an affected skin area, said method comprising the steps of applying to said affected inflamed skin area a composition comprising sertaconazole and wherein said affected skin area is not infected.

A kit for treating a pruritis skin condition comprising an affected skin area that itches, said kit comprising an applicator and a composition comprising sertaconazole, wherein said affected skin area is not infected.

A kit for treating an inflamed skin condition comprising an affected skin area, said kit comprising an applicator and a composition comprising sertaconazole, wherein said affected skin area is not infected.

A composition for treating a pruritis skin condition comprising an affected skin area that itches, said composition comprising sertaconazole, wherein said affected skin area is not infected.

A composition for treating an inflamed skin condition comprising an affected skin area, said composition comprising sertaconazole, wherein said affected skin area is not infected.

### DETAILED DESCRIPTION OF THE PREFERRED EMBODIMENTS

One of the objects of this invention is to provide compositions, methods and kits for providing rapid relief to the user and for treating pruritic skin disorders.

Another of the objects of this invention is to provide compositions, methods and kits for treating neurogenic skin disorders.

Still another object of this invention is the prevention of pruritic and neurogenis inflammatory skin disorders.

As used herein, the term “irritation”, includes all types of itching (pruritic and non-pruritic itching), stinging, burning, tingling, “tightness”, erythema (redness) or edema (swelling).

As used herein, the term “pruritic” includes itching that is often associated with urticaria, the eruption of a skin rash, which may consist of red spots, scaly patches, or blisters.

As used herein, the term “neurogenic inflammation”, also referred to as neurosensory inflammation, includes those types of inflammation such as those triggered by sensory nerve activation in skin. Certain natural substances, temperature and protons act on small diameter sensory neurons C-fibers, the nerve fibers that conduct pain, itch and stinging sensations. Activation of small diameter sensory neurons induces release inflammatory neuropeptides such as substance P and calcitonin gene-related peptide. These substances, in turn, act on peripheral blood vessels and immune cells producing an inflammatory response that is characterized by erythema, edema, warmth and hypersensitivity (Richardson J and Vasko M R, J. Pharm Exp Therap. 302:839-845, 2002) In mouse skin, an edema response occurs rapidly upon application of a vanilloid receptor activator, such as capsaicin or resiniferatoxin (RTX). Some skin diseases or conditions may be induced or exacerbated by neurogenic inflammation. Such conditions include eczema, psoriasis, acne, rosacea, contact irritant dermatitis, atopic dermatitis, cold and heat induced urticaria, allergic dermatitis, sunlight-induced dermatoses and dry skin (Holzer P, Gen Pharmacol. 30:5-11, 1998). Compounds that inhibit the response to could be useful as topical analgesics, itch or sting inhibitors or soothing agents for irritated skin (see U.S. Pat. No. 6,090,811). It is well recognized that activity of a compound in inhibiting cellular mediated inflammation is not predictive of activity to inhibit neurogenic inflammation or itch (see U.S. Pat. No. 6,090,811 or Inoue H, et al., Br J Pharmacol. 110:1614-1620, 1993. The irritation response may be due to the direct effect on the skin of certain topical product chemicals, a response by the immune system directed toward the chemicals alone or in combination with skin components (e.g., allergic dermatitis), or to a skin disease or disorder.

As used herein, the term non-infected skin conditions refers to those skin conditions that exhibit irritation and itching wherein the skin is not infected and the conditions are not the result of an infection. By the word infection, it shall include common skin infections caused by bacterial, viral or fungal organisms. In one example of an infected skin condition, vaginal yeast infections typically result in itching and irritation of the perineum. Those types of infections are not the subjects of this treatment. Pruritic and neurogenic conditions are those that are included in this invention. For example, itching as a result of a contact dermatitis is included as typically, no infection is present.

As previously mentioned, shaving disrupts the skin's barrier function. Not only do people shave their faces, legs and underarms, some people shave the groin area. This area can become inflamed with ingrown hair.

In one embodiment of the invention, the composition used to treat the affected skin area contains Sertaconazole.

In another embodiment, the composition is applied to a skin area to prevent the skin from becoming affected.

The composition used in this invention may be in the form of a cream, ointment, lotion, or gel. In one embodiment, the composition is an oil-in-water emulsion.

The composition, methods and kits of this invention may contain other ingredients.

In addition to the an-itching agent, the composition may include an ameliorating agent.

As used herein, the term ameliorating agent, may include local anesthetics or compounds that abolishes the sensation of pain and thereby provides relief. Examples of ameliorating agents include local anesthetics, antihistamines, anti-inflammatories, skin protectants and those ingredients that provide a cooling sensation.

Local anesthetics and antihistamines that are useful in the compositions of this invention include pramoxine, benzocaine, lidocaine, dibucaine, benzyl alcohol, camphor, resorcinol, menthol and diphenhydramine hydrochloride and the like.

Anti-inflammatories such as corticosteroids, including hydrocortisone acetate, may also be employed in the external topical compositions of this invention. COX 2 Inhibitors may also be used, such as Valdecoxib, Celocoxib and Refecoxib. Non-steroidal anti-inflammatory drugs (NSAIDS) such as Indomethacin, Naproxen Sodium, Naproxen Potassium, Diclofenac sodium, Oxaproxin, Salicylate, Etodolac, Meloxicam, Ketoprofen, Tolmecytin sodium, Choline Magnesium and Trisalicylate may also be useful in the compositions and devices of this invention.

External topical compositions of this invention may also contain skin protectants. By protecting the skin, not only does the composition soothe the site of infection; it also maintains the integrity of the skin to prevent additional damage and pain. Skin protectants may include allantoin, cocoa butter, dimethicone, kaolin, shark liver oil, petrolatum, vegetable oils, zinc oxide and others known to those of skill in the art.

Other beneficial ingredients or other chemical agents may also be included in order to convey to the patient a sensation of cooling. Sensations such as cooling may provide the perception of relief to the user, especially if the inflamed area is infected. These beneficial ingredients include lower alcohols, menthol, camphor, sorbitol, sugars such as monosaccharides, disaccharides, oligosaccharides, polysaccharides, plant extracts such as aloe, witch hazel, chamomile, hydrogenated soy oil and colloidal oatmeal, and vitamins such as vitamins A, D or E or the like may also be. included.

As used herein, the term “active agents” shall include those ingredients such as those having antimicrobial activity not including antifungal properties.

In one embodiment, the composition of this invention includes an active agent such as an antimicrobial. Such antimicrobials includes, but are not limited to, antibacterials, antivirals, antibiotics, and the like.

In another embodiment of the invention, the composition includes one or more antimicrobial agents. The antimicrobial may be chosen from the group including, but not limited to, metronidazole, clindamycin, tinidazole, ornidazole, secnidazole, refaximin, trospectomycin, and their pharmaceutically acceptable salts and the like.

Antiviral active ingredients include Acyclovir, emtricitabine, ribavirin, adefovir, dipivioxil, tenefovir, retrovir, epivir, indinavir, lamivudin, emetricitabine, sequnavir, hydroxyurea, fosamprenavir and the like.

Another embodiment of the compositions of this invention include one or more antiviral agents. Antiviral agents may also include, but are not limited to, immunomodulators, more preferably imiquimod, its derivatives, podofilox, podophyllin, interferon alpha, reticolos, cidofovir, nonoxynol-9 and their pharmaceutically acceptable salts and the like.

Other components may be present in the composition of this invention such as water, anti-oxidants, chelating agents, preservatives, oils, waxes, surfactants, emulsifiers, viscosity building agents, buffering agents, solvents, moisturizing agents, solubilizers and bioadhesives/muco-adhesives and then like. The relative quantities of such components may vary according to the desired nature and consistency of the composition. The composition may be in any free flowing form, including, but not limited to, suspensions, emulsion, clear and opaque gels, semi-solid systems, including ointments, pastes, oil-in-water creams, semi-solid emulsions with solid internal phases, semi-solid emulsions with fluid internal phases, lotions and the like.

Preferred embodiments include those in which the compositions of this invention are in the form of a cream. In one embodiment, the cream is an oil-in-water emulsion. In one embodiment, the composition containing sertaconazole is used for treating a pruritis skin condition wherein said affected skin area is not infected. In another embodiment, the composition containing sertaconazoleis used for treating an inflamed skin condition wherein said affected skin area is not infected.

In one embodiment of the invention, a patient suffering from a skin condition, which causes itching, for example, applies to the affected or itching area a composition containing sertaconazole. In another embodiment, a patient who is pre-disposed to skin conditions, may apply sertaconazole to prevent future skin conditions such as pruritis.

In another embodiment of the invention, a patient suffereing from neurogenic skin inflammation applies to the affected skin area a composition containing sertaconazole. In another embodiment, a patient who is predisposed to skin neurogenic skin inflammation applies to the skin area, a composition containing sertaconazole to prevent future inflammation. Agents or skin conditions that induce pruritis are transduced by a subgroup of sensory neurons, identified as nociceptors, that give rise to itch sensations (Schmelz M. and Handwerker HO., Neurophysiologic Basis of Itch, in Itch Basic Mechanisms and Therapy, ed. Yosipovitch G., et al, 2003). Inhibition of sensory neuron signal transduction prior to pruritic stimulation can prevent pruritic and neurogenic responses. Prevention of pruritic or neurogenic inflammation can be demonstrated by topical pretreatment of the composition into an uninfected region of skin for 1-2 days prior to induction with an agent(s) that induces a pruritic or neurogenic response. Inhibition of the pruritic or neurogenic response resulting from a pretreatment regiment can be used to establish the ability of the composition to prevent pruritis or neurogenic inflammation.

In one embodiment of the invention, a patient suffering a skin condition, which causes pruritis, uses a kit containing an applicator and composition containing sertaconazole. In one embodiment, the applicator is a tube having a closed end and delivery portion. The delivery end may have a cap. The patient would remove the cap, expel the composition out of the delivery portion onto the skin. The patient may then rub the composition into the skin, coating the affected area.

In another embodiment of the invention, a patient suffering from neurogenic skin inflammation, uses a kit containing an applicator and composition containing sertaconazole. In one embodiment, the applicator is a tube having a closed end and delivery portion. The delivery end may have a cap. The patient would remove the cap, expel the composition out of the delivery portion onto the skin. The patient may then rub the composition into the skin, coating the affected area.

The following examples serve to illustrate, but not limit, the scope of the inventions described herein.

### EXAMPLE 1

Eight Albino male CD-1 mice per group, 7-9 weeks old, were used. Induction of neurogenic inflammation in the mouse ear was based on known methods (Inoue H, et al., Br J Pharmacol. 110:1614-1620, 1993). A 20-μl volume of Resiniferatoxin (0.05%) prepared in acetone was applied to the left ears (8 mice per treatment group). The right ear was treated as a control and not treated. Lidocaine hydrochloride (Sigma Aldrich, St. Louis, Mo.) was prepared as a 0.5% w/v solution in 70% ethanol/30% propylene glycol vehicle. Sertaconazole nitrate (Grupo Ferrer International, Barcelona, Spain) was prepared as a 1.0% w/v solution in a 70% ethanol/30% propylene glycol vehicle and applied to the left ear (20 μL) immediately after resiniferatoxin challenge. The mice were sacrificed by CO₂ inhalation 30 minutes after applying the solutions. The left and right ears were removed and a 7-mm biopsy was removed from each ear and weighed. The difference in biopsy weights between the right and left ear was calculated. The percent inhibition was calculated by comparing treatments to resiniferatoxin alone. Anti-neurogenic inflammation effects of compounds are evident as an inhibition of the increase in ear weight.

| | Topical Dose, | % Inhibition of |
|---|---|---|
| Treatment | as % w/v | the Ear Edema* |
| Lidocaine | 0.5 | 28.7 |
| Sertaconazole | 1 | 48.9 |
| Nitrate | | |

*The % Inhibition (Percent Inhibition) is equal to the ((Vehicle Biopsy Weight-Treatment Biopsy Weight)/Vehicle Biopsy Weight) × 100

Sertaconazole was highly effective in this model in reducing neurogenic inflammation when compared to the control or untreated ear.

### EXAMPLE 2

In this model, an itch-associated response is induced by intradermal injection of the neuropetide, Substance-P, in mice, and scratching behavior is observed and quantitated. Substance-P is a undecapeptide belonging to the tachykinin family and intradermal injection of Substance-P has been shown to elicit pruritogenic (itch) responses in humans. Clinically, scratching is used as an objective measure of itch since itch is a sensation which promotes the desire to scratch the stimulated area (Yosipovitch G., Definitions of Itch, in Itch Basic Mechanisms and Therapy, ed. Yosipovitch G., et al, 2003)

Eight Albino male CD-1 mice per group, 7-9 weeks old, were used. Induction of scratching behavior in the mouse was based on known methods (Andoh T, et al., J Pharmacol Exp Ther. 286:1140-1145, 1998) and produces features similar to itch in humans. An itch-associated response was induced by intradermal injection of Substance P in CD-1 mice, and scratching behavior is observed and quantitated. Before the experiment, mice were individually housed in a plastic cage for at least 1 hour for acclimation. Mice are pre-treated for 30 minutes with topical application of Sertaconazole Nitrate (1%) prepared in 100% ethanol, to an area of the back, which had been shaved one day prior to the experiment. Substance P is prepared in sterile physiological saline and a total of 300 ug of Substance P is injected in a volume of 50 uL into the interscapular part of the back. Injection of sterile physiological saline alone as a control did not result in induction a significant scratch response. After injection, mice were returned to the cage and their scratching behaviors videotaped. The number of scratches elicited during a 30 minute period after injection of Substance-P was determined by playback of the videotape.

| | | Scratches per |
|---|---|---|
| | Topical Dose, | 30 min |
| Treatment | as % w/v | (Mean ± S. D.) |
| Vehicle | 0 | 65.0 ± 8.1 |
| Sertaconazole | 1 | 39.8 ± 2.7 |
| Nitrate | | |

Sertaconazole was highly effective in this model reducing the itch by 38.7% compared to placebo when compared to the control.

### EXAMPLE 3

This example illustrates an Oil-in-water emulsion incorporating sertaconazole.

Preparation of Preparation of Oil Phase:

200.0 g of Pegoxol-7 stearate available from Gattefosse under the tradename “Tefose-63”, 80.0 g of Light Mineral Oil NF available from Holland Applied Technologys under the tradename “Drakeol”, 50.0 g of PEG-6 palm kernel oil available from Gattefosse under the tradename “Labrafil M 2130”, and 20.0 g of Glyceryl Monooleate available from Gattefosse under the tradename “Peceol” were combined into a primary glass beaker and heated to 70-75° C. while mixed at moderate speed with a lightning mixer until melted and uniform. 1.0 g of Sorbic Acid available from City Chemical was added and stirred until homogeneous at a temperature of 70-75° C.

Preparation of Water Phase:

628.0 g of deionized water were weighed into a primary glass beaker and heated to 70-75° C. While mixed at moderate speed with a lightning mixer, 1.0 g of Methylparaben available-from NIPA laboratories was added and mixed until homogenous.

Preparation of Final Composition:

Adjust the temperature of the Oil Phase and the Aqueous Phase to between 70° C. and 80° C. Add the Aqueous Phase to the Oil Phase and begin mixing to homogenize. Continue mixing for 10-15 minutes. Cool to between 35° C. and 45° C., add 20.0 g of Sertaconazole Nitrate then mix for 10-15 minutes or until uniform. Cool to 25° C. The resultant composition is a spreadable cream.

What is claimed is:

1. A method of treating a pruritis skin condition comprising an affected skin area that itches, said method comprising the steps of applying to said affected skin area a composition comprising sertaconazole and wherein said affected skin area is not infected.

2. A method of treating an inflamed skin condition comprising an affected skin area, said method comprising the steps of applying to said affected inflamed skin area a composition comprising sertaconazole and wherein said affected skin area is not infected.

3. A kit for treating a pruritis skin condition comprising an affected skin area that itches, said kit comprising an applicator and a composition comprising sertaconazole, wherein said affected skin area is not infected.

4. A kit for treating an inflamed skin condition comprising an affected skin area, said kit comprising an applicator and a composition comprising sertaconazole, wherein said affected skin area is not infected.

5. A composition for treating a pruritis skin condition comprising an affected skin area that itches, said composition comprising sertaconazole, wherein said affected skin area is not infected.

6. A composition for treating an inflamed skin condition comprising an affected skin area, said composition comprising sertaconazole, wherein said affected skin area is not infected.

7. A method of claim 1 further comprising an ameliorating agent.

8. A method of claim 7, wherein the ameliorating agent comprises a local anesthetic.

9. A method of claim 8, wherein the ameliorating agent comprises an antihistamine.

10. A method of claim 1, further comprising a pharmaceutically acceptable carrier.

11. A method of claim 1, further comprising an active agent.

12. A method of claim 1, further comprising a cooling ingredient.

13. A method of claim 12, wherein the cooling ingredient comprises from about 5 to about 35% w/w of a polyol.

14. A method of claim 11, wherein the active agent is an antibiotic.

15. A method of claim 14, wherein the antibiotic is selected from the group consisting of: metronidazole, clindamycin, tinidazole, ornidazole, secnidazole, refaximin, trospectomycin, purpuromycin and their pharmaceutically acceptable salts.

16. A method of claim 11, wherein the active agent is an antiviral compound.

17. A method of claim 16, wherein said antiviral compound is selected from the group consisting of: Acyclovir, emtricitabine, ribavirin, adefovir, dipivioxil, tenefovir, retrovir, epivir, indinavir, lamivudin, emetricitabine, sequnavir, hydroxyurea and fosamprenavir.

18. A method of claim 12, wherein the cooling compound comprises lower alcohols, menthol, camphor and sugars.
